(19) United States
(12) Patent Application Publication
(10) Pub. No.: US 2013/0183261 A1
(43) Pub. Date: Jul. 18, 2013

(54) METHOD AND COMPOSITION FOR DELIVERING ACTIVE INGREDIENT INTO AIR, AND USE THEREOF

(71) Applicants: Akiko Harada, Takarazuka-shi (JP); Timothy C. Hadingham, Wallingford (GB)

(72) Inventors: Akiko Harada, Takarazuka-shi (JP); Timothy C. Hadingham, Wallingford (GB)

(73) Assignee: SUMITOMO CHEMICAL COMPANY, LIMITED

(21) Appl. No.: 13/814,429

(22) PCT Filed: Aug. 3, 2011
(86) PCT No.: PCT/JP2011/068212

§ 371 Date: Mar. 19, 2013

(30) Foreign Application Priority Data
Aug. 6, 2010 (JP) 2010-177976

(51) Int. Cl. A61L 9/01; A61L 9/14

(52) U.S. Cl. CPC A61L 9/01; A61L 9/14
USPC 424/76.2; 422/4; 422/123; 568/679; 512/5; 512/1; 428/34.1

(57) ABSTRACT

The present invention is directed to a method and a spray composition for delivering an active ingredient into air. The spray composition comprises a carrier composition containing a glycol ether having a structure represented by the following General Formula: R¹O—[CH₂CH(CH₃)O]n-H (R¹ is C₁-C₄ alkyl, n is 2 or 3 (when n is 2, R¹ is not methyl)), and an active ingredient mixed with the carrier composition, wherein the carrier composition has a vapour pressure of less than 14 Pa at 20° C. The method comprises the step of spraying the spray composition into air, and, optionally, the step of preparing a spray composition by mixing the active ingredient with the carrier composition.

### TECHNICAL FIELD

The present invention relates to a method and a spray composition for delivering an active ingredient into air.

### BACKGROUND ART

In daily lives, there are many occasions when it is desirable to treat the air. For example, it may be beneficial to deliver a fragrance into the air to mask or to eliminate malodours. Alternatively, it may be beneficial to introduce into the air small amounts of microbiocidal active ingredients in order to sanitize micro-organisms in the air.

In air treatment, sprays of liquids are convenient means to deliver active ingredients into the air. Often, sprays of liquid have an added advantage of accelerating the removal of airborne contaminants from the air by agglomeration with chemical species such as dust, pollen, airborne allergens and smoke particles.

Further desirable properties of air treatment formulations are compatibility with regulations relating to products containing Volatile Organic Compounds (VOCs). For example, the General Consumer Products Regulation (Article 2) (the Regulation) in the US State of California controls the sale, supply, offers for sale or manufacture of consumer products which at the time of sale or manufacture, contain VOCs in excess of specified limits.

The term VOC is known to those skilled in the art and methods for determining the VOC content include those referenced in the Regulation and elsewhere (for example using Air Resource Board Method 310 “Determination of Volatile Organic Compounds (VOC) in Consumer Products” (ARB Method 310)). VOC are organic chemical compounds that boil in a range up to approximately 260° C. at normal pressure.

The VOC content of liquid spray air treatment compositions suitable for aerosol, piezoelectric, liquid electric wicks and electrostatic spray devices are substantially determined by the non-aqueous solvents of the compositions. Often, the water content of a composition is increased so as to reduce the relative contribution of non-aqueous solvents. This practice imposes severe limitations on the variety and scope of active ingredients that may be included in an air treatment composition and may also detrimentally affect the performance in spray devices such as those noted above.

Examples of formulations for treating the air are known in the art, particularly for treating the air by using electrostatic spray, but are generally unsuited to dispersal into the air where regulations restrict VOC content of formulations, have unsuitable physical properties for low volume spray dispersal (unsuitable viscosity and/or resistivity), have unpredictable purities of constituents (e.g. vegetable oils) or have undesirable toxicological properties, for example by requiring surfactants to maintain a single phase, or are otherwise harmful to humans and the environment.

Patent Literatures 1 and 2 are directed to formulations delivered by electrostatic spraying. The compositions include “Exsol” D180/220, “Solvesso” 150, Aromasol H, kerosene (which typically have unsuitable VOC content), groundnut oil, and soybean oil (which typically have unsuitable physical properties and/or inconsistent purities) as solvents.

Patent Literature 3 discloses compositions comprising oleic acid, whose vapour pressure would make the compositions unsuitable according to VOC regulations.

Patent Literature 4 discloses solvents for electrostatic spray including dimethyl isosorbide and propylene glycol, which have unsuitable vapour pressures at 20° C., and glycerol and polyethylene glycols with undesirable physical properties for low volume electrostatic spray where there is no mechanical pumping to overcome high viscosity.

Patent Literature 5 discloses electrostatic spray formulations comprising ethanol, perfluorooctanol, and perfluorodecalin (which have high vapour pressure) or water (which has high surface tension).

Patent Literature 6 discloses compositions for electrostatic spray comprising isopropyl alcohol, phenyl ethyl glycol, 1,3-butanediol, 1,2-propanediol which have too high a vapour pressure, unsuitable physical properties, undesirable toxicological properties, are not odourless, or combinations of these properties.

Patent Literature 7 relates to liquid compositions for dispersion by electrostatic spray and discloses bulk solvents including cyclohexanone and combinations of glycol solvents comprising at least one lower alkanol (which have unsuitable vapour pressures).

Patent Literature 2 discloses compositions particularly suited to ultra-low volume electrostatic spraying. Bulk solvents disclosed include “Solvesso” 150, “Isopar” L and “Exsol” D180/220, the resistivities of which are high but taught to be lowered by mixing with alcohols and ketonic solvents, which may be unsuitable for safety reasons.

Patent Literature 8 discloses compositions suited to electrostatic spray comprising glycol ethers, 3-methoxy-3-methyl butanol, isododecane, diethylphthalate, and isopropyl myristate (which have unsuitable vapour pressures), but does not identify solvents that would be suitable for low VOC regulations.

### CITATION LIST

Patent Literature 1
European Patent Application Publication No. 0066946, specification

Patent Literature 2
European Patent Application Publication No. 0003251, specification

Patent Literature 3
European Patent Application Publication No. 0006293, specification

Patent Literature 4
European Patent Application Publication No. 0224352, specification

Patent Literature 5
International Application Publication No. 00/066206, pamphlet

Patent Literature 6
European Patent Application Publication No. 0523960, specification

Patent Literature 7
Australian patent No. 592970, specification

Patent Literature 8
International Application Publication No. 03/000431, pamphlet

### SUMMARY OF INVENTION

### Solution to Problem

A first aspect of the present invention provides a method for delivering an active ingredient into air, comprising the step of spraying a spray composition into air. In the method, the spray composition comprises:

a carrier composition containing glycol ether having a structure represented by the following General Formula (I)

R¹O—[CH₂CH(CH₃)O]ₙ—H
wherein R¹ is an alkyl group having 1-4 carbon atoms, n is 2 or 3, and when n is 2, R¹ is not a methyl group; and

an active ingredient mixed with the carrier composition,

the carrier composition having a vapour pressure of less than 14 Pa at 20° C. The carrier composition may further comprise a dibasic ester and/or dipropylene glycol. Further, the spray composition may further comprise a resistivity-modifying component and, optionally, a surface tension-modifying component.

Further, a second aspect of the present invention provides a spray composition for delivering an active ingredient into air.

Further, a third aspect of the present invention provides use of glycol ether being represented by the General Formula (I) or the carrier composition, for delivering an active ingredient into air.

Further, a fourth aspect of the present invention provides a reservoir containing the spray composition.

Further, a fifth aspect of the present invention provides a spray device comprising a reservoir containing the spray composition, a spray electrode connected with the reservoir so as to receive the spray composition, a discharging electrode in a vicinity of the spray electrode, and an application section for applying an electric field across the spray electrode and the discharging electrode, upon application of an electric field across the spray electrode and the reference electrode, the spray composition being sprayed from the spray electrode in the form of droplets.

### Advantageous Effects of Invention

The present invention provides a method and a composition for air treatment which complies with VOC regulations and can stably deliver an optional active ingredient into air, and which is especially suitable for spray by an electrostatic spray device. To be specific, the present invention provides a method and a composition for air treatment which poses low toxicity and low hazards to humans and the environment and avoids the use of undesirable constituents such as propellants and ethanol.

### DESCRIPTION OF EMBODIMENTS

<Composition>

A composition of the present invention comprises:

a carrier composition containing glycol ether having a structure represented by the following General Formula (I)

R¹O—[CH₂CH(CH₃)O]ₙ—H
wherein R¹ is an alkyl group having 1-4 carbon atoms, n is 2 or 3, and when n is 2, R¹ is not a methyl group; and

an active ingredient mixed with the carrier composition,

the carrier composition having a vapour pressure of less than 14 Pa at 20° C. The carrier composition may further comprise a dibasic ester and/or dipropylene glycol. Further, the spray composition may further comprise a resistivity-modifying component and, optionally, a surface tension-modifying component.

In the specification and the claims, “vapour pressure” indicates “vapour pressure determined by CARB method 310 (Air Resources Board Method 310) for a composition from which any aqueous component is removed”.

Further, All references including patent and patent applications referred to in this application are incorporated herein by reference to the fullest extent possible.

A preferable example of the composition of the present invention is such that contents of a low vapour pressure component a), a resistivity-modifying component b), a active ingredient c), and a surface tension-modifying component d) are present in the following ranges.

TABLE 1

| | | |
|---|---|---|
| a) Low vapour pressure component | 55-99.45% | w/w |
| b) Resistivity-modifying component | 0.5-5% | w/w |
| c) Active ingredient | 0.05-44.5% | w/w |
| d) Surface tension-modifying component | 0-10% | w/w |

In the specification, the term “low vapour pressure component” indicates a component which was found by the inventors of the present invention to be capable of realizing the present invention, and is an essential component of the carrier composition.

In the composition of the present invention, the low vapour pressure component a) comprises glycol ether having a structure represented by the General Formula (I), and optionally a dibasic ester and/or dipropylene glycol.

That is, the low vapour pressure component includes glycol ether having a structure represented by the General Formula (I) as an essential component, and may include a dibasic ester and/or dipropylene glycol as an optional component.

In the General Formula (I) representing the glycol ether, R¹ is an alkyl group having 1-4 carbons. The alkyl group may be a straight chain one, a branched chain one, or cyclic one. The alkyl group is preferably a straight chain one or a branched chain one. Specific examples of the alkyl group include a methyl group, an ethyl group, an n-propyl group, an isopropyl group, an n-butyl group, an isobutyl group, a sec-butyl group, a tert-butyl group.

In the General Formula (I), n is 2 or 3. It should be noted that when n is 2, R¹ is not a methyl group.

Examples of the glycol ether represented by the General Formula (I) include dipropylene glycol ethyl ether, dipropylene glycol n-propyl ether (which may be hereinafter abbreviated as DPnP), dipropylene glycol n-butyl ether, tripropylene glycol methyl ether, tripropylene glycol ethyl ether, tripropylene glycol n-propyl ether, and tripropylene glycol n-butyl ether. Among them, dipropylene glycol n-propyl ether is preferable.

They may be used alone or in combination.

Examples of the dibasic ester (which may be hereinafter abbreviated as DBE) include dimethyl glutarate and dimethyl adipate.

They may be used alone or in combination.

In the present invention, the dibasic ester is preferably at least one selected from dimethyl glutarate and dimethyl adipate.

In a case where the dibasic ester is a mixture of dimethyl glutarate and dimethyl adipate, a ratio of dimethyl glutarate in the mixture preferably ranges from 5 to 80% by weight, and a ratio of dimethyl adipate in the mixture preferably ranges from 20 to 95% by weight.

In the composition of the present invention, the lower limit of the content of the low vapour pressure component a) is preferably 55%, more preferably 57.5% and particularly preferably 59% by weight of the total composition. The upper limit of the content of the low vapour pressure component a) is 99.45%, preferably 90%, and most preferably 89% by weight of the total composition. The content of the low vapour pressure component a) preferably lies between any of these upper and lower limits.

In a case where the composition of the present invention contains the glycol ether represented by the General Formula (I), the lower limit of the content of the glycol ether is preferably 35%, more preferably 40% and particularly preferably 42% by weight of the total composition. The upper limit of the content of the glycol ether is preferably 90%, more preferably 88%, and particularly preferably 87.5% by weight of the total composition. The content of the glycol ether preferably lies between any of these upper and lower limits.

In a case where the composition of the present invention contains the dibasic ester, the lower limit of the content of the dibasic ester is preferably 5%, more preferably 6% and particularly preferably 12% by weight of the total composition. The upper limit of the content of the dibasic ester is preferably 85%, more preferably 84.5%, and particularly preferably 36% by weight of the total composition. The content of the dibasic ester preferably lies between any of these upper and lower limits.

In a case where the composition of the present invention contains dipropylene glycol, the lower limit of the content of dipropylene glycol is preferably 1%, more preferably 4% and particularly preferably 4.4% by weight of the total composition. The upper limit of the content of dipropylene glycol is preferably 15%, more preferably 10%, and particularly preferably 9% by weight of the total composition. The content of dipropylene glycol preferably lies between any of these upper and lower limits.

The resistivity-modifying component b) may be a component known to those skilled in the art, capable of controlling the resistivity of the composition. Examples thereof include water and electrolyte.

Generally, water contains minimal electrolyte. The resistivity of water is typically at least 1×10¹ Ωm, more typically at least 1×10³ Ωm, particularly typically at least 1×10⁴ Ωm and most typically at least 1×10⁵ Ωm. For example, it is often that the resistivity is at least 1×10⁶ Ωm or at least 10×10⁷ Ωm.

Water used in the present invention preferably has purity equal to or higher than that of deionized water or water for High Performance Liquid Chromatography (HPLC).

The electrolyte may be added, in the form of an aqueous solution for example, to the composition. Examples of the electrolyte include sodium acetate, sodium hydrogen carbonate, sodium chloride, ascorbic acid, citric acid and acetic acid. In a case where the composition of the present invention contains the electrolyte, a ratio of the content of the electrolyte to the content of aqueous component in the composition is preferably 0.1-2% and more preferably 0.1-1% by weight.

The resistivity-modifying component may be used alone or in combination.

In the composition of the present invention, the lower limit of the content of the resistivity-modifying component b) is preferably 0.5%, more preferably 0.7% and particularly preferably 1% by weight of the total composition. The upper limit of the content of the resistivity-modifying component b) is preferably 5%, more preferably 4.5%, and particularly preferably 4% by weight of the total composition. The content of the resistivity-modifying component b) preferably lies between any of these upper and lower limits.

In the present invention, the active ingredient c) may be any component according to the purpose. Preferable examples of the active ingredient c) include fragrances and air sanitizers.

Examples of the fragrances include essential oils and other fragrance oils. The fragrances may be only a part of all fractions (component oils) of these oils.

Preferable examples of the fragrances include Tea tree oil (e.g. oil of Melaleuca, Terpinen-4-ol type), Catmint oil (e.g. oil of Nepeta cateria, refined oil of Nepeta cateria) and fractions thereof (e.g fractions comprising nepetalactone), Thyme oil (e.g. oil of Thymus vulgaris) and fractions thereof (e.g. fractions comprising thymol).

Fragrances such as fragrance oils are often mixtures containing plural kinds of components with different skeleton chain lengths or mixtures containing plural kinds of stereoisomers. These mixtures may be used.

Among them, the fragrant material is preferably an oil component of at least one oil selected from the group consisting of tea tree oil, Catmint oil, and Thyme oil.

In a case where the composition of the present invention contains the fragrance oil, the content of the fragrance oil is preferably 5-35% by weight of the total composition.

The active ingredient c) preferably contains 55-95%, more preferably 57.5-90%, and further preferably 59-78% by weight of the fragrance oil.

The fragrance oil preferably has a vapour pressure of 270 Pa or less at 20° C.

Examples of the air sanitizers include active air-cleaner ingredients, active air freshener ingredients, active anti-microbial ingredients, active anti-fungal ingredients and active anti-allergenic ingredients. To be more specific, preferable examples of the air sanitizers include polyhexamethylene bigauanide polymer, polyhexamethyl gaunide polymer, alkyl dimethyl benzyl ammonium chloride, octyl decyl dimethyl ammonium chloride, chlorhexidine, chlorhexidine digluconate, benzalkonium chloride, sodium hypochlorite, 2-phenylphenol, polyethylene glycol 300, 2-benzyl-4-chlorophenol, 2-phenoxyethanol, glutaraldehyde, phthalaldehyde, chloroxylenol, trichlorophenol, phenol, silver salts (especially water-soluble silver salts), hexachlorophene, peracetic acid, lactic acid, performic acid, potassium permanganate, and potassium peroxymonosulphate.

In a case where the composition of the present invention contains the air sanitizer, the content of the air sanitizer is preferably 0.05-20%, more preferably 0.1-17%, and particularly preferably 0.1-15% by weight of the total composition.

Examples of the active anti-microbial ingredients include triclosan, trichloro carbanilide, isoprophyl methyl phenol, N-(dichlorofluoromethyltio)-phthalic amide, N′-(dichlorofluoromethyltio)N,N′-dimethyl-N′-phenyl-sulfamide, polyoctyl polyaminoethylglycine, thiabendazole, chlorine dioxide, 2-bromo-2-nitroethanol, 2-bromo-2-nitropropane-1,3-diol, 2-bromo-2-nitropropanol, 1-bromo-1-nitropropanol, 1,4-dibromo-1,4-dinitrobutanediol-2,3-cetylpyridinium, 1-bromo-1-nitro-2-methylpropanol-2-cetylpyridinium and cetylpyridinium chloride, benzethonium chloride, acrinol, povidoniodine, mercurochrome, chloramphenicol, fradiomycin sulfate, gentamicin sulfate, oxytetracycline chloride, polymyxin B sulfate, trichomycin, and griseofulvin.

Examples of the active anti-fungal ingredients include: benzoic acid and salt thereof, sorbic acid and salt thereof, paraoxy benzoic esters, sodium dehydroacetate, propionic acid, polylysine, thiabendazole; terpene alcohols such as linalool, geraniol, nerol, citronellol, α-terpineol, terpinene-4-ol, and isopulegol; alicyclic alcohols having 7-15 carbons such as 2,4-dimethyl-3-cyclohexane-1-methanol, 4-isopropylcychlohexanol, 4-isopropylcyclohexanemethanol, 1-(4-isopropylcyclehexyl)-ethanol, 2,2-dimethyl-3-(3-methylphenyl)-propanol; and arylalkyl alcohols or alkylaryl alcohols having 7-15 carbon atoms such as benzyl alcohol, phenylethyl alcohol, phenylpropyl alcohol, carvacrol, and eugenol.

Examples of the active anti-allergenic ingredients include hydroxyapatite, epicatechin, epigallocatechin, epicatechin gallate, epigallocatechin gallate, gallic acid, and an ester compound of gallic acid and alcohol having 1-4 carbon atoms.

Examples of the active air-cleaner ingredients include polyphenol such as tannin and flavonoid (chalcone, flavanone, flavanol, flavone, flavonol, and isoflavone), cyclodextrin, lauryl methacrylate, geranylchlorinate, 4-hydroxy-6-methyl-3-(4-methyl pentanoyl)-2-pyrone, formalin, glyoxal, sodium bisulfite, sodium sulfite, dihydroxyacetone, 3,5,5-trimethylhexanol, β-etoxypropionaldehyde, glutaraldehyde, methacrylic ester, maleic ester, maleic monoamide, maleic imide, humaric ester, β-acyl acrylic acid and salt thereof, citronellol senecionate, 1-3-pentadiene-1-carboxylic alkylester, pinane hydroperoxide, p-cymene peroxide, 1,2-propyleneoxide, 1,2-butyleneoxide, glycidyl ether, saccharose octaacetate, Fe(III)-octacarboxyphthalocyanine, Fe(III)-tetracarboxyphthalocyanine, 5-methyl-2-isopropyl-2-hexenal, p-butoxyphenol, catechol, hydroquinone, 4-methylcatechol, 1,2,4-trihydroxybenzene, 3-methylcatechol, 3-methoxycatechol, carnosol, rosmanol, brazilin, hematoxylin, shikonin, myricetin, baicalein, baicalin, citral, vanillin, and coumarin.

Preferable examples of the active ingredient c) further include a one which also serves as the resistivity-modifying component b). Examples of such active ingredient c) include the salts of quaternary amines, preservatives, salts of chlorhexidine including chlorhexidine digluconate, and other air sanitizers described above. Such active ingredient c) may be used alone while also serving as the resistivity-modifying component b) or in combination with other active ingredient c) or the resistivity-modifying component b).

The active ingredient c) may be used alone or in combination.

In a case where two or more active ingredients c) are used together, the two or more active ingredients c) may be two or more kinds of fragrances, or may be two or more kinds of air sanitizers, or may include at least one fragrance and at least one air sanitizer.

In the composition of the present invention, the content of the active ingredient c) is preferably 0.05 to 44.5% by weight of the total composition.

The composition of the present invention may contain, optionally, the surface tension-modifying component d) as an optional component, in addition to the low vapour pressure component a), the resistivity-modifying component b), and the active ingredient c).

Surface tension-modifying component d) is added to the composition of the present invention in order to lower the surface tension of the bulk formulation or in order that the aforementioned physical properties of the composition are closer to a required range.

Examples of the surface tension-modifying component d) include isoparaffin (e.g. Isopar L); and silicone oils such as decamethyltetrasiloxane, decamethylcyclopentasiloxane, dodecamethylcyclohexasiloxane, and mixtures thereof.

The surface tension-modifying component d) may be used alone or in combination.

In a case where the composition of the present invention contains the surface tension-modifying component d), the lower limit of the content of the surface tension-modifying component d) is preferably 0.5%, more preferably 1%, particularly preferably 2%, and most preferably 3% by weight of the total composition. The upper limit of the content of the surface tension-modifying component is preferably 10%, more preferably 8%, and most preferably 4% by weight of the total composition. The content of the surface tension-modifying component d) preferably lies between any of these lower limits and upper limits.

The composition of the present invention may contain other components in addition to the low vapour pressure component a), the resistivity-modifying component b), the active ingredient c), and the surface tension-modifying component d).

Examples of such other components include a viscosity-modifying component. For example, it is possible to increase the viscosity of the composition by adding glycerine.

Further, a small amount of an optical tracer may be contained in the composition of the present invention provided that the optical tracer does not affect the overall nature of the composition. For example, Uvitex-OB etc. in an amount up to 1% by weight may be contained.

An example of the composition of the present invention which contains fragrance oil as the active ingredient c) contains

55-89.5% w/w low vapour pressure component a);
0.5-5% w/w resistivity-modifying component b);
10 to 35% w/w fragrance oil; and
0-8% w/w surface tension-modifying component d).

An example of the composition of the present invention which contains the air sanitizer as the active ingredient contains (and preferably consists essentially only of)

70-97.9% w/w low vapour pressure component a);
1-5% w/w resistivity-modifying component b);
0.1 to 20% w/w air sanitizer agent; and
1-10% w/w surface tension-modifying component d).

An example of the composition of the present invention which contains the fragrance oil and the air sanitizer as the active ingredient c) contains (and preferably consists essentially only of)

55-92.9% w/w low vapour pressure component a);
1-5% w/w resistivity-modifying component b);
5 to 35% w/w fragrance oil;
0.1 to 20% w/w air sanitizer agent;
1-10% w/w surface tension-modifying component d).

The relative proportions of the active ingredient c) together with any low vapour pressure component a), resistivity-modifying component b) and surface-tension-modifying component d) may conveniently be adjusted within the above stated ranges such that the resistivity, surface tension and vapour pressure of the final composition is appropriate in order that a stable spray of appropriately-sized droplets for efficient dispersal of the active ingredient is obtained.

Preferable physical properties of the composition of the present invention are as follows.

The resistivity at 20° C. ranges preferably from 1×10³ to 1×10⁶ Ωm, and more preferably from 1.5×10³ to 8×10⁴ Ωm.

The surface tension at 20° C. ranges preferably from 20 to 40 mN/m, and more preferably from 26 to 34 mN/m.

The viscosity at 20° C. ranges preferably from 1 to 10 mPa·s, and more preferably from 2 to 5 mPa·s.

It is preferable that the composition of the present invention has at least two or all of these physical properties.

Measurement of the physical properties of the composition of the present invention is known to those in the art. The ways in which the physical properties are measured are not particularly limited. Specific examples thereof are as follows.

The resistivity of the composition may be measured by using a liquid resistive cell. The surface tension of the composition may be measured using the Du Noüy Ring method, providing measurements in mN/m. The viscosity of the composition may be measured using a viscometer, providing measurements in mPa·s and the physical size (the diameter) of droplets of a sprayed composition may be measured using an aerodynamic particle sizer, providing measurements in microns. The vapour pressure was determined following CARB 310 method.

Compositions for air treatment according to the prior art typically contain solvents with unacceptable amounts of VOCs for the health of humans and the environment or otherwise have unacceptable toxicological profiles or are otherwise too viscous for effective delivery into the air without using a pump. One advantage of the present invention is that compositions according to the present invention are free or substantially free of VOCs or are at least within permitted levels according to CARB regulations (The California Air Resources Board's regulations on VOC).

The composition of the present invention allows the consistent generation of small droplets which are substantially clear and monodisperse (e.g. range in size between 0.1-20 μm, for example droplets of volume median diameter less than 10 microns) and retain a non-zero residual charge when employed in an electrostatic spray device (such as that disclosed in European Patent Application Publication No. 1399265 specification). The size of the droplet can be measured by a publicly known method such as a method using an aerodynamic particle sizer, and the measured value is on the μm basis.

The composition of the present invention is highly versatile with application to a wide range of active ingredient c), including those not generally thought to be miscible with organic solvents (such as water-soluble air sanitizers (e.g. the salts of quaternary amines and other ionic active ingredients)) and for which hitherto formulation with substantially aqueous compositions was required.

A conventional composition to be sprayed contains propellant whose amount will influence the properties of the composition. Here, Propellants are low boiling point substances (i.e. liquefied gases) which have appreciable vapour pressure at ambient temperature, e.g. substances which have a boiling point of less than 0° C. (e.g. less than −40° C.) at atmospheric pressure (1.025×10⁵ Pa). Example propellants to be avoided include lower hydrocarbons such as propane and butane. Other example propellants to be avoided include CFCs (chlorofluorocarbons) such as Propellant 12 and HFAs (hydrofluoroalkanes) such as HFA134a and HFA227.

In contrast thereto, the composition of the present invention does not contain a propellant as an essential component. A content of the propellant in the composition is typically less than 5%, more typically less than 1%, and particularly typically 0.1% by weight. The content may be 0% by weight. No propellant or substantially no propellant is contained. The propellant content will have essentially no impact on the properties of the composition.

The composition of the present invention does not contain, as an essential component, ethanol, propylene glycol methyl ether, dipropylene glycol methyl ether, ethylene glycol or propylene glycol which have high vapour pressure.

The ethanol, propylene glycol methyl ether, dipropylene glycol methyl ether, ethylene glycol or propylene glycol content in the composition of the present invention is typically less than 5%, more typically less than 1%, and particularly typically less than 0.1% by weight. The ethanol, propylene glycol methyl ether, dipropylene glycol methyl ether, ethylene glycol or propylene glycol content may be 0% by weight, and is not contained substantially or at all. Ideally the amount of ethanol, glycol methyl ether, ethylene glycol or propylene glycol will be undetectable in the composition once dispensed (i.e. be undetectable by taste or smell).

The composition of the present invention does not require a chemical material having inappropriate physical properties such as glycerol and polyethylene glycol, and so may be dispersed without mechanical pumping.

Suitably the composition of the invention should remain within the above stated physical properties window (i.e., in terms of resistivity, surface tension and vapour pressure) over the entire range of temperatures expected during normal operation. The “entire range of temperatures” here indicates a range of temperatures within which a liquid composition for air treatment is normally used. Specifically, this range is appropriately 0-40° C., typically a room temperature, and more typically 18-25° C., and particularly typically approximately 20° C.

Dipropylene glycol n-propyl ether (DPnP) for example has a high viscosity (11.4 mPa·s at 25° C.), which would be expected to require mechanical assistance of electrostatic spray dispersal to achieve a suitable delivery rate. However, surprisingly, DPnP is suitable for dispersal according to the present invention without mechanical assistance of a mechanical pump etc., even when present in the composition at high amounts (e.g. greater than 85% w/w).

The surface tension of dibasic ester (DBE) is relatively high (e.g. 38.8 mN/m) and it has very little water solubility (water in solvent 2.5 wt % at 20° C.), making it unsuitable for co-formulation with active ingredients requiring aqueous solvation or resistivity-modifying components (which are typically weakly electrolytic solutions) without resorting to emulsifying agents. Therefore, it has been surprising that DBE is suitable for formulation in the composition of the present invention without further requirement of emulsifiers. Furthermore, the high surface tension of DBE would be expected to result in an unacceptably low delivery rate. However, surprisingly, the composition of the present invention provides stable flow rates of between 0.4-1.0 g formulation/day for example, and so is particularly suitable for electrostatic spray. Furthermore, compositions according to the invention generally have flash points above 64° C., thereby reducing hazards in manufacture, handling and transportation. Other advantages embodied by the compositions of the invention include the fact that the compositions are less-flammable and have higher flash point than solvents of the art, thus representing less of a fire risk (by contrast with prior art compositions which contain ethanol and/or low molecule weight hydrocarbons and/or glycol methyl ether, ethylene glycol or propylene glycol), thus representing less of a fire risk and do not need to be stored under pressure.

[Method for Treating Air, Use of Composition, and Method for Delivering Fixed Quantity of Active Ingredient]

A method of the present invention for treating air includes spraying the composition of the present invention into the air.

A method of the present invention for delivering fixed quantity of active ingredient includes taking the composition of the present invention and spraying it from an electrostatic spray device.

As described above, the composition of the present invention is suitable for being delivered by spray, and can be delivered at a stable flow rate for a long time. The delivery may be made occasionally or continuously.

Air treatment can be made by taking the composition of the present invention contained in a reservoir for example and spraying it into the air.

The spray device is preferably an electrostatic spray device. An example thereof is an electrostatic spray device of the present invention including a reservoir containing the composition, a spray electrode into which the composition is fed from the reservoir, and a reference electrode in the vicinity of the spray electrode, upon application of an electric field across the spray electrode and the reference electrode, the composition being sprayed from the spray electrode in the form of droplets.

In an electrostatic spray device, a composition is fed to a spray electrode, and the spray electrode is ultimately exposed to the electric field and the electric field (potential difference) is applied between the spray electrode and a reference electrode, so that the composition is sprayed in the form of droplets. Such an electrostatic spray device is well known in the art as described by Sir Geoffrey Taylor in the Proceedings of the Royal Society—1964, pages 383-397.

The electrostatic spray device described in European Patent Application Publication No. 1399265 specification is suitable for use of the composition of the present invention. This provides a simple, robust spray device with a spray electrode located near the outlet surface of the device, with another reference and discharging electrode in close proximity, also located near the device outlet surface. When configured in an open geometry, both electrodes are generally protected from user interference by sheltering inside separate recesses in the outlet surface. The outlet surface comprises a dielectric material, which is selected such that it leaks charge at a slow rate so that any charge deposited by way of ions or charged particles does not migrate or leak away immediately. This ensures that these dielectric recesses retain a slight charge of the same polarity as the electrodes they house. This significantly alters the local electric field shape and ensures that further deposition is discouraged—most significantly without the need for extra electrodes. A further advantage is that the device is polarity independent. In other words, the spray electrode can be at any voltage (positive or negative) and the discharging electrode at any other voltage—provided that only the potential difference is sufficient to create the spray in the first instance. The range of workable potential differences depends on the distance between the two electrodes, the depth to which they are recessed from the outlet surface and the size of the recesses themselves, and potential differences can range from 1-2 kV, up to 30 kV or more and can be both positive or negative in relative polarity.

In one possible embodiment of the device described in European Patent Application Publication No. 1399265 specification, a spray electrode is housed inside a spray recess and a discharging electrode is housed inside a discharging recess. The spray electrode in this example comprises a 27-gauge metal or conductive plastic capillary and the discharging electrode comprises a sharp, stainless steel pin, 0.6 mm in diameter.

The two longitudinal axes of the recesses are perpendicular to a spray outlet surface, which is manufactured from a dielectric material. In this embodiment, the material is nylon and the spray outlet surface is flat. However, other materials and curved surfaces can be used provided that there is sufficient charge retention at the spray outlet surface to deflect the spray and charge carriers away from the device and electrodes. Electrical connections connect to a driving circuit.

In the electrostatic spray device, the composition to be sprayed is held in a reservoir and air is fed into the reservoir to replace the sprayed liquid via the small air inlet hole.

When this device is energised by a driving circuit, the composition from the reservoir is emitted from the spray electrode in a very fine form, such that it quickly evaporates according to its vapour pressure and the ambient conditions in the vicinity of the device. Use of the composition of the present invention in an electrostatic spray device is also encompassed in the present invention.

An electrostatic spray device may be adapted to deliver the composition of the present invention in an occasional manner (e.g. using a duty cycle) or continuously.

The following explains the present invention in more details with reference to specific Examples. It should be noted that the present invention is not limited to the Examples below.

Fragrance oils A, B, and C used in preparing the composition in the Examples are fragrance oils 225696, 163047, 180262, respectively from Firmenich S. A., Route des Jeunes 1, P.O. Box 239, Geneve 8 CH-1211, Switzerland (“Firmenich”). Fragrance oil D is fragrance oil Tea tree oil CAS No. 68647-73-4 conforming to ISO 4730 (2004) “Oil of melaleuca, Terpinen-4-ol type” from Firmenich.

[Preparation of Composition and Evaluation of Physical Properties of Composition]

### Example 1

The composition as shown in Table 2 was prepared and measured in terms of its resistivity, surface tension, flash point, and vapour pressure, and the physical properties of the composition were evaluated. The result of the evaluation is shown in Table 2.

TABLE 2

| a) Low vapour pressure | DPnP | 42% w/w |
|---|---|---|
| component | Mixture of dimethyl | 18% w/w |
| | glutarate and dimethyl | |
| | adipate | |
| | (mixture ratio 30:70 | |
| | (w/w)) | |
| b) Resistivity-modifying | Sodium acetate | 0.012% w/w |
| component | Water | 2.988% w/w |
| c) Active ingredient | Fragrance oil A | 30% w/w |
| | Polyethylene glycol 300 | 3% w/w |
| d) Surface | Isopar L | 4% w/w |
| tension-modifying | | |
| component | | |
| | Total | 100% |

| | |
|---|---|
| Resistivity | 3.9 × 10³ Ωm |
| Surface tension | 32.5 mN/m |
| Flash point | 70° C. |
| Vapour pressure | 12 Pa at 20° C. |

### Example 2

The composition shown in Table 3 was prepared and measured in terms of its resistivity, surface tension, and flash point, and vapour pressure of the low vapour pressure component a), and the physical properties of the composition were evaluated. The result of the evaluation is shown in Table 3.

TABLE 3

| | | | |
|---|---|---|---|
| a) Low vapour pressure | DPnP | 42% | w/w |
| component | Mixture of dimethyl | 20% | w/w |
| | glutarate and dimethyl | | |
| | adipate | | |
| | (mixture ratio 30:70 | | |
| | (w/w)) | | |
| b) Resistivity-modifying | Sodium acetate | 0.012% | w/w |
| component | Water | 2.988% | w/w |
| c) Active ingredient | Fragrance oil B | 30% | w/w |
| | Polyethylene glycol 300 | 5% | w/w |

| | | |
|---|---|---|
| d) Surface | n/a | n/a |
| tension-modifying | | |
| component | | |
| | Total | 100% |

| | |
|---|---|
| Resistivity | 1.6 × 10⁴ Ωm |
| Surface tension | 32 mN/m |
| Flash point | 75° C. |
| Vapour pressure | 8.0 Pa at 20° C. |

### Example 3

The composition shown in Table 4 was prepared and measured in terms of its resistivity, surface tension, and flash point, and vapour pressure of the low vapour pressure component a), and the physical properties of the composition were evaluated. The result of the evaluation is shown in Table 4.

TABLE 4

| | | | |
|---|---|---|---|
| a) Low vapour pressure | DPnP | 59% | w/w |
| component | | | |
| b) Resistivity-modifying | Sodium acetate | 0.016% | w/w |
| component | Water | 3.984% | w/w |
| c) Active ingredient | Fragrance oil C | 30% | w/w |
| | Polyethylene glycol 300 | 3% | w/w |
| d) Surface | Isopar L | 4% | w/w |
| tension-modifying | | | |

| | | |
|---|---|---|
| component | | |
| | Total | 100% |

| | |
|---|---|
| Resistivity | 7.9 × 10³ Ωm |
| Surface tension | 30.9 mN/m |
| Flash point | 65° C. |
| Vapour pressure | 9.3 Pa at 20° C. |

### Example 4

The composition shown in Table 5 was prepared and measured in terms of its resistivity, surface tension, and flash point, and vapour pressure of the low vapour pressure component a), and the physical properties of the composition were evaluated. The result of the evaluation is shown in Table 5.

TABLE 5

| | | | |
|---|---|---|---|
| a) Low vapour pressure | DPnP | 47% | w/w |
| component | Mixture of dimethyl | 31% | w/w |
| | glutarate and dimethyl | | |
| | adipate | | |
| | (mixture ratio 30:70 | | |
| | (w/w)) | | |
| b) Resistivity-modifying | Sodium acetate | 0.016% | w/w |
| component | Water | 3.984% | w/w |
| c) Active ingredient | Fragrance oil D | 10% | w/w |
| | Polyethylene glycol 300 | 5% | w/w |
| d) Surface | Isopar L | 3% | w/w |
| tension-modifying | | | |

| | | |
|---|---|---|
| component | | |
| | Total | 100% |

| | |
|---|---|
| Resistivity | 1.8 × 10³ Ωm |
| Surface tension | 32 mN/m |
| Flash point | 66° C. |
| Vapour pressure | 9.3 Pa at 20° C. |

### Example 5

The composition shown in Table 6 was prepared and measured in terms of its resistivity, surface tension, and flash point, and vapour pressure of the low vapour pressure component a), and the physical properties of the composition were evaluated. The result of the evaluation is shown in Table 6.

TABLE 6

| | | | |
|---|---|---|---|
| a) Low vapour pressure | DPnP | 46.2% | w/w |
| component | Mixture of dimethyl | 30.8% | w/w |
| | glutarate and dimethyl | | |
| | adipate | | |
| | (mixture ratio 30:70 | | |
| | (w/w)) | | |
| b) Resistivity-modifying | Sodium acetate | 0.016% | w/w |
| component | Water | 3.984% | w/w |
| c) Active ingredient | Fragrance oil D | 10% | w/w |
| | Polyethylene glycol 300 | 5% | w/w |
| | 2-phenylphenol | 1% | w/w |
| d) Surface | Isopar L | 3% | w/w |
| tension-modifying | | | |

| | | |
|---|---|---|
| component | | |
| | Total | 100% |

| | |
|---|---|
| Resistivity | 3.2 × 10³ Ωm |
| Surface tension | 32 mN/m |
| Flash point | 66° C. |
| Vapour pressure | 9.3 Pa at 20° C. |

### Example 6

The composition shown in Table 7 was prepared and measured in terms of its resistivity, surface tension, and flash point, and vapour pressure of the low vapour pressure component a), and the physical properties of the composition were evaluated. The result of the evaluation is shown in Table 7.

TABLE 7

| | | | |
|---|---|---|---|
| a) Low vapour pressure | DPnP | 53.4% | w/w |
| component | Mixture of dimethyl | 35.6% | w/w |
| | glutarate and dimethyl | | |
| | adipate | | |
| | (mixture ratio 30:70 | | |
| | (w/w)) | | |
| b) Resistivity-modifying | Sodium acetate | 0.012% | w/w |
| component | Water | 2.988% | w/w |
| c) Active ingredient | Polyethylene glycol 300 | 5% | w/w |
| d) Surface | Isopar L | 3% | w/w |
| tension-modifying | | | |

| | | |
|---|---|---|
| component | | |
| | Total | 100% |

| | |
|---|---|
| Resistivity | 7.9 × 10³ Ωm |
| Surface tension | 32.2 mN/m |
| Flash point | 72° C. |
| Vapour pressure | 9.3 Pa at 20° C. |

### Example 7

The composition shown in Table 8 was prepared and measured in terms of its resistivity, surface tension, and flash point, and vapour pressure of the low vapour pressure component a), and the physical properties of the composition were evaluated. The result of the evaluation is shown in Table 8.

TABLE 8

| | | | |
|---|---|---|---|
| a) Low vapour pressure | DPnP | 82% | w/w |
| component | Dipropylene glycol | 5% | w/w |
| b) Resistivity-modifying | Sodium acetate | 0.004% | w/w |
| component | Water | 0.996% | w/w |
| c) Active ingredient | Chlorhexidine digluconate | 1% | w/w |
| | Polyethylene glycol 300 | 3% | w/w |
| d) Surface | Isopar L | 8% | w/w |
| tension-modifying | | | |
| component | | | |

| | |
|---|---|
| Total | 100% |

| | |
|---|---|
| Resistivity | 3.1 × 10⁴ Ωm |
| Surface tension | 30.1 mN/m |
| Flash point | 64° C. |
| Vapour pressure | 4.0 Pa at 20° C. |

The compositions of Examples 1-7 produce stable spray with efficient dispersion of the active component without observable deposition when used with an electrostatic spray device disclosed in European Patent Application Publication No. 1399265 specification for a continuous period of 1 day spraying with a 20.9% duty cycle at a flow rate of around 0.40-1.0 g of formulation/day.

### Comparative Example 1

The composition shown in Table 9 was prepared and measured in terms of its resistivity, surface tension, and flash point, and vapour pressure of the non-low vapour pressure component, and the physical properties of the composition were evaluated. The result of the evaluation is shown in Table 9.

TABLE 9

| | | | |
|---|---|---|---|
| Non-low vapour | Dipropylene glycol | 88% | w/w |
| pressure component | methyl ether | | |
| b) Resistivity-modifying | Sodium acetate | 0.008% | w/w |
| component | Water | 1.992% | w/w |
| c) Active ingredient | Polyethylene glycol | 5% | w/w |
| | 300 | | |
| d) Surface tension- | Isopar L | 5% | w/w |

| | | |
|---|---|---|
| modifying component | | |
| | Total | 100% |

| | |
|---|---|
| Resistivity | 1.57 × 10⁴ Ωm |
| Surface tension | 30.7 mN/m |
| Flash point | 62° C. |
| Vapour pressure | 39 Pa at 20° C. |

### Comparative Example 2

The composition shown in Table 10 was prepared and measured in terms of its resistivity, surface tension, and flash point, and vapour pressure of the non-low vapour pressure component, and the physical properties of the composition were evaluated. The result of the evaluation is shown in Table 10.

TABLE 10

| | | | |
|---|---|---|---|
| Non-low vapour | Dipropylene glycol | 60% | w/w |
| pressure component | methyl ether | | |
| b) Resistivity-modifying | Sodium acetate | 0.012% | w/w |
| component | Water | 2.988% | w/w |
| c) Active ingredient | Fragrance oil A | 30% | w/w |
| | Polyethylene glycol | 3% | w/w |
| | 300 | | |
| d) Surface | Isopar L | 4% | w/w |
| tension-modifying | | | |

| | | |
|---|---|---|
| component | | |
| | Total | 100% |
| Resistivity | | 7.85 × 10³ Ωm |
| Surface tension | | 30.5 mN/m |
| Flash point | | 70° C. |
| Vapour pressure | | 40 Pa at 20° C. |

The compositions of Comparative Examples 1 and 2 are conventional compositions comprising solvents according to the art with suitable characteristics of resistivity, viscosity and surface tension for electrostatic spray. These compositions exhibit vapour pressures which are greater than 14 Pa and are therefore unsuited to dispersal into the air where regulations restrict VOC content of formulations.

In a preferred aspect, the present invention provides a composition and a method suitable for delivering an active component into the air, which complies with VOC regulations, which is capable of stably delivering an optional active ingredient into the air, and which is especially suitable for spraying by an electrostatic spray.

It is an object of the invention to provide a composition suitable for delivering an active component into the air, especially by dispersal in the air in aerosol sprays and more especially by electrostatic spray. It is a further object of the invention to provide a composition which poses low toxicity and low hazards to humans upon dispensing of the composition and otherwise avoids the use of undesirable constituents such as propellants and ethanol. It is a further object of the invention to provide a composition which is clear and exists in a single phase. It is a further object of the invention to provide a composition suitable for treating the air which is also low in volatile organic compounds (VOC). It is a further object of the invention that the composition is capable of stably supplying an optional active ingredient or ingredients such as a fragrance oil or a sanitisation agent or combinations thereof when sprayed.

These and other objects of the invention are achieved by providing a composition and a method according to the invention.

The present invention provides a method and a spray composition for delivering an active ingredient into air, and provides use of a specific compound or a carrier composition for delivering an active ingredient into air.

The method of the present invention may be arranged such that a total content of the glycol ether and, optionally, the dibasic ester and/or dipropylene glycol is 55-99.45% w/w of the spray composition.

The method of the present invention may be arranged such that the spray composition has at 20° C. a resistivity in a range of 1×10³ to 1×10⁶ Ωm and a surface tension in a range of 20 to 40 mN/m.

The method of the present invention may be arranged such that a content of the active ingredient is 0.05-44.5% w/w, a content of the resistivity-modifying component is 0.5-5% w/w, and a content of the surface tension-modifying component is 0-10% w/w.

The method of the present invention may be arranged such that the glycol ether is selected from the group consisting of tripropylene glycol monomethyl ether, tripropylene glycol n-butyl ether, dipropylene glycol n-propyl ether, and dipropylene glycol n-butyl ether.

The method of the present invention may be arranged such that the dibasic ester is dimethyl glutarate and/or dimethyl adipate.

The method of the present invention may be arranged such that the spray composition further comprises a viscosity-modifying component.

The method of the present invention may be arranged so as to further include the step of preparing the spray composition by mixing the active ingredient with the carrier composition.

The method of the present invention may be arranged such that the active ingredient is a fragrance.

The method of the present invention may be arranged such that the fragrance is a fragrance oil.

The method of the present invention may be arranged such that the fragrance oil is selected from Tea tree oil, Catmint oil, Thyme oil.

The method of the present invention may be arranged such that the fragrance oil is present at 5-35% w/w.

A method of the present invention for delivering an active ingredient into air comprises the step of spraying a spray composition into air,

the spray composition comprising:
glycol ether represented by General Formula (I)

R¹O—[CH₂CH(CH₃)O]ₙ—H
wherein R¹ is an alkyl group having 1-4 carbon atoms, n is 2 or 3, and when n is 2, R¹ is not a methyl group; and
optionally, a dibasic ester and/or dipropylene glycol,

the spray composition further comprising a resistivity-modifying component, a fragrance oil, and a surface tension-modifying component,

a total content of the glycol ether, and optionally, the dibasic ester and/or dipropylene glycol being 55-89.5% w/w of the spray composition, and

a content of the resistivity-modifying component being 0.5-5% w/w, a content of the fragrance oil being 10-35% w/w, and a content of the surface tension-modifying component being 0-8% w/w.

The method of the present invention may be arranged such that the active ingredient is an air sanitizer.

The method of the present invention may be arranged such that the air sanitizer is present at 0.05-20% w/w.

A method for delivering an active ingredient into air, comprising the step of spraying a spray composition into air,

the spray composition comprising:
glycol ether represented by General Formula (I)

R¹O—[CH₂CH(CH₃)O]ₙ—H
wherein R¹ is an alkyl group having 1-4 carbon atoms, n is 2 or 3, and when n is 2, R¹ is not a methyl group; and
optionally, a dibasic ester and/or dipropylene glycol,

the spray composition further comprising a resistivity-modifying component, an air sanitizer, and a surface tension-modifying component,

a total content of the glycol ether, and optionally, the dibasic ester and/or dipropylene glycol being 70-97.9% w/w of the spray composition, and

a content of the resistivity-modifying component being 1-5% w/w, a content of the air sanitizer being 0.1-20% w/w, and a content of the surface tension-modifying component being 1-10% w/w.

The method of the present invention may be arranged such that the step of spraying is carried out electrostatically.

The method of the present invention may be arranged so as to further include the steps of:

feeding the spray composition to a spray electrode for spraying the spray composition into air; and

applying an electric field between the spray electrode and a discharge electrode in a vicinity of the spray electrode.

The use of the present invention may be arranged such that the glycol ether is electrostatically sprayed in combination with the active ingredient so that the active ingredient is delivered into air.

The use of the present invention may be arranged such that the glycol ether is used in combination with a dibasic ester and/or dipropylene glycol.

The use of the present invention may be arranged such that the glycol ether is used in combination with a resistivity-modifying component and/or a surface tension-modifying component, and optionally, a viscosity-modifying component.

In another aspect, the present invention provides a method and a composition suitable for treating the air, which complies with VOC regulations, which is capable of stably delivering an optional active ingredient into the air, and which is especially suitable for spraying by an electrostatic spray.

It is an object of the invention to provide a composition suitable for treating the air, especially by dispersal in the air in aerosol sprays and more especially by electrostatic spray. It is a further object of the invention to provide a composition which poses low toxicity and low hazards to humans upon dispensing of the composition and otherwise avoids the use of undesirable constituents such as propellants and ethanol. It is a further object of the invention to provide a composition which is clear and exists in a single phase. It is a further object of the invention to provide a composition suitable for treating the air which is also low in volatile organic compounds (VOC). It is a further object of the invention that the composition is capable of stably supplying an optional active ingredient or ingredients such as a fragrance oil or a sanitisation agent or combinations thereof when sprayed.

These and other objects of the invention are achieved by providing a composition according to the invention.

Thus, a liquid composition of the present invention for treating the air comprises:

a) a low vapour pressure component comprising at least one compound selected from (i) and (ii), wherein

(i) is a compound represented by General Formula (I):

R¹O—[CH₂CH(CH₃)O]ₙ—H; and

(ii) is a dibasic ester;

wherein the low vapour pressure component optionally further comprises dipropylene glycol;

b) a resistivity-modifying component;

c) at least one active ingredient; and optionally

d) a surface tension-modifying component;

wherein the low vapour pressure component a) of the composition has at 20° C. a vapour pressure of less than 14 Pa.

The composition of the present invention may be arranged such that the low vapour pressure component a) content of the composition is 55-99.45% w/w.

The composition of the present invention may be arranged such that the low vapour pressure component a) content of the composition is 57.5-90% w/w.

The composition of the present invention may be arranged such that the low vapour pressure component a) content of the composition is 59-89% w/w.

The composition of the present invention may be arranged such that the low vapour pressure component a) comprises a component selected from the list consisting of: tripropylene glycol monomethyl ether, tripropylene glycol n-butyl ether, dipropylene glycol n-propyl ether, dipropylene glycol n-butyl ether and dibasic esters.

The composition of the present invention may be arranged such that the low vapour pressure component a) comprises dipropylene glycol n-propyl ether.

The composition of the present invention may be arranged such that the low vapour pressure component a) comprises a dibasic ester component.

The composition of the present invention may be arranged such that the dibasic ester component is selected from the list consisting of: dimethyl glutarate, dimethyl adipate.

The composition of the present invention may be arranged such that the dibasic ester component is a mixture of dimethyl glutarate and dimethyl adipate.

The composition of the present invention may be arranged such that the dibasic ester component content is 5-85% w/w.

The composition of the present invention may be arranged such that the at least one active ingredient comprises a fragrance.

The composition of the present invention may be arranged such that the fragrance is a fragrance oil.

The composition of the present invention may be arranged such that the fragrance comprises a component oil selected from Tea tree oil, Catmint oil, Thyme oil.

The composition of the present invention may be arranged such that the component oil is a fraction of the total oil present.

The composition of the present invention may be arranged such that the fragrance oil is present at 5-35% w/w.

The composition of the present invention may be arranged so as to include:

55-89.5% w/w low vapour pressure component a);

0.5-5% w/w resistivity modifying component b);

15-35% w/w fragrance oil;

0-8% w/w surface tension-modifying component c).

The composition of the present invention may be arranged such that the at least one active ingredient comprises an air sanitizer.

The composition of the present invention may be arranged such that the air sanitizer is present at 0.05-20% w/w.

The composition of the present invention may be arranged so as to include:

70-97.9 w/w low vapour pressure component a);

1-5% w/w resistivity-modifying component b);

0.1-20% w/w air sanitizing agent;

1-10% w/w surface tension-modifying component c).

The composition of the present invention may be arranged such that the composition further comprises a viscosity-modifying component.

The composition of the present invention having at 20° C. a resistivity in the range 1×10³ to 1×10⁶ Ωm and a surface tension in the range 20-40 mN/m.

The composition of the present invention may be arranged so as to be an electrostatic spray composition.

A method of treating the air uses the composition of the invention.

A spray device reservoir of the present invention contains a composition according to the first aspect of the invention.

The spray device reservoir of the present invention may be arranged such that the spray device is an electrostatic spray device.

An electrostatic spray device of the present invention comprises a spray electrode into which the composition to be sprayed is fed from a reservoir, a reservoir containing composition to be sprayed (being a composition according to the first aspect of the invention), and a reference electrode in the vicinity of the spray electrode, such that upon application of electric potential across the electrodes the composition is sprayed from the spray electrode in the form of droplets.

Use of the present invention is use of the aforementioned composition in an electrostatic spray device.

A process of the present invention for dispensing an active ingredient comprises taking a composition according to the first aspect of the invention and delivering it from an electrostatic spray device.

A surprising aspect of the invention is that the use of at least one compound selected from (i) a compound represented by General Formula (1) in the composition (as defined herein), (e.g. dipropylene glycol n-propyl ether (DPnP)) and (ii) a dibasic ester, and optionally in combination with dipropylene glycol provides a liquid which is suitable for air treatment when sprayed into the air, preferably when using an electrostatic spray device. Whereas glycol ethers have long been described in the art as suitable for electrostatic spray treatments of the air, we have found that this has been a gross oversimplification since compositions comprising these solvents contain unacceptable amounts of VOCs for the health of humans and the environment or otherwise have unacceptable toxicological profiles or are otherwise too viscous for effective delivery into the air without the need for expensive pump devices.

Suitably the composition of the invention should remain within the above stated physical properties window (i.e., in terms of resistivity, surface tension and vapour pressure) over the entire range of temperatures expected during normal operation. More often than not this is room temperature (say between 18 and 25° C. e.g. at 20° C.) which is therefore the optimum temperature range. However, the compositions disclosed under this invention will typically operate from 0° C. to above 40° C. without modification, and often also outside this range.

Non-aqueous solvents of the art are unsuitable for satisfying relevant VOC conditions and it is an object of the present invention to provide compositions of non-aqueous solvents which, among other objects, satisfy relevant VOC conditions such that a component of a composition according to the invention comprises less than (substance) % w/w of substances with a vapour pressure greater than 14 Pa at 20° C. (where the amount (substance) % is determined according to product category as defined by the Regulation), as determined by ARB method 310. Any fragrance oil component which may be present in the formulation may have a vapour pressure of less than or equal to 280 Pa at 20° C.

1. A method for delivering an active ingredient into air, comprising the step of spraying a spray composition into air,
the spray composition comprising:
a carrier composition containing glycol ether having a structure represented by General Formula (I)
R¹O—[CH₂CH(CH₃)O]ₙ—H
wherein R¹ is an alkyl group having 1-4 carbon atoms, n is 2 or 3, and when n is 2, R¹ is not a methyl group; and
an active ingredient mixed with the carrier composition,
the carrier composition having a vapour pressure of less than 14 Pa at 20° C.

2. The method according to claim 1, wherein the carrier composition further comprises a dibasic ester and/or dipropylene glycol.

3. The method according to claim 1, wherein a total content of the glycol ether and, optionally, the dibasic ester and/or dipropylene glycol is 55-99.45% w/w of the spray composition.

4. The method according to claim 1, wherein the spray composition further comprises a resistivity-modifying component and, optionally, a surface tension-modifying component.

5. The method according to claim 4, wherein the spray composition has at 20° C. a resistivity in a range of 1×10³ to 1×10⁶ Ωm and a surface tension in a range of 20 to 40 mN/m.

6. The method according to claim 5, wherein a content of the active ingredient is 0.05-44.5% w/w, a content of the resistivity-modifying component is 0.5-5% w/w, and a content of the surface tension-modifying component is 0-10% w/w.

7. The method according to claim 1, wherein the glycol ether is selected from the group consisting of tripropylene glycol monomethyl ether, tripropylene glycol n-butyl ether, dipropylene glycol n-propyl ether, and dipropylene glycol n-butyl ether.

8. The method according to claim 1, wherein the dibasic ester is dimethyl glutarate and/or dimethyl adipate.

9. The method according to claim 1, wherein the spray composition further comprises a viscosity-modifying component.

10. The method according to claim 1, further comprising the step of preparing the spray composition by mixing the active ingredient with the carrier composition.

11. The method according to claim 1, wherein the active ingredient is a fragrance.

12. The method according to claim 11, wherein the fragrance is a fragrance oil.

13. The method according to claim 12, wherein the fragrance oil is selected from Tea tree oil, Catmint oil, Thyme oil.

14. The method according to claim 12, wherein the fragrance oil is present at 5-35% w/w.

15. A method for delivering an active ingredient into air, comprising the step of spraying a spray composition into air,
the spray composition comprising:
glycol ether represented by General Formula (I)
R¹O—[CH₂CH(CH₃)O]ₙ—H
wherein R¹ is an alkyl group having 1-4 carbon atoms, n is 2 or 3, and when n is 2, R¹ is not a methyl group; and
optionally, a dibasic ester and/or dipropylene glycol,
the spray composition further comprising a resistivity-modifying component, a fragrance oil, and a surface tension-modifying component,
a total content of the glycol ether, and optionally, the dibasic ester and/or dipropylene glycol being 55-89.5% w/w of the spray composition, and
a content of the resistivity-modifying component being 0.5-5% w/w, a content of the fragrance oil being 10-35% w/w, and a content of the surface tension-modifying component being 0-8% w/w.

16. The method according to claim 1, wherein the active ingredient is an air sanitizer.

17. The method according to claim 16, wherein the air sanitizer is present at 0.05-20% w/w.

18. A method for delivering an active ingredient into air, comprising the step of spraying a spray composition into air,
the spray composition comprising:
glycol ether represented by General Formula (I)
R¹O—[CH₂CH(CH₃)O]ₙ—H
wherein R¹ is an alkyl group having 1-4 carbon atoms, n is 2 or 3, and when n is 2, R¹ is not a methyl group; and
optionally, a dibasic ester and/or dipropylene glycol,
the spray composition further comprising a resistivity-modifying component, an air sanitizer, and a surface tension-modifying component,
a total content of the glycol ether, and optionally, the dibasic ester and/or dipropylene glycol being 70-97.9% w/w of the spray composition, and
a content of the resistivity-modifying component being 1-5% w/w, a content of the air sanitizer being 0.1-20% w/w, and a content of the surface tension-modifying component being 1-10% w/w.

19. The method according to claim 1, wherein the step of spraying is carried out electrostatically.

20. The method according to claim 19, further comprising the steps of:
feeding the spray composition to a spray electrode for spraying the spray composition into air; and
applying an electric field between the spray electrode and a discharge electrode in a vicinity of the spray electrode.

21. Use of glycol ether for delivering an active ingredient into air,
the glycol ether being represented by General Formula (I)
R¹O—[CH₂CH(CH₃)O]ₙ—H
wherein R¹ is an alkyl group having 1-4 carbon atoms, n is 2 or 3, and when n is 2, R¹ is not a methyl group.

22. The use according to claim 21, wherein the glycol ether is electrostatically sprayed in combination with the active ingredient so that the active ingredient is delivered into air.

23. The use according to claim 21, wherein the glycol ether is used in combination with a dibasic ester and/or dipropylene glycol.

24. The use according to claim 21, wherein the glycol ether is used in combination with a resistivity-modifying component and/or a surface tension-modifying component, and optionally, a viscosity-modifying component.

25. A reservoir, containing a spray composition comprising:
a carrier composition containing glycol ether represented by General Formula (I)
R¹O—[CH₂CH(CH₃)O]ₙ—H
wherein R¹ is an alkyl group having 1-4 carbon atoms, n is 2 or 3, and when n is 2, R¹ is not a methyl group; and
an active ingredient mixed with the carrier composition,
the carrier composition having a vapour pressure of less than 14 Pa at 20° C.

26. A spray device, comprising:
a spray electrode connected with a reservoir according to claim 25 so as to receive the spray composition;
a discharging electrode in a vicinity of the spray electrode; and
an application section for applying an electric field across the spray electrode and the discharging electrode,
the electric field being applied across the spray electrode and the discharging electrode so that the spray composition fed from the reservoir into the spray electrode is sprayed into air at the spray electrode.
